(19) United States
(12) Patent Application Publication
(10) Pub. No.: US 2022/0059225 A1
(43) Pub. Date: Feb. 24, 2022

(54) SYSTEM AND METHODOLOGY FOR DIAGNOSIS, PREDICTION AND TREATMENT OF AMYGDALA HYPERACTIVITY IN HUMAN PATIENTS

(71) Applicant: Kamal Ramesh Shah, Katy, TX (US)

(72) Inventor: Kamal Ramesh Shah, Katy, TX (US)

(21) Appl. No.: 17/408,460

(22) Filed: Aug. 22, 2021

Related U.S. Application Data
(60) Provisional application No. 63/069,013, filed on Aug. 22, 2020.

(51) Int. Cl. G16H 50/20; G16H 40/67; G16H 10/60; G16H 20/70; G16H 20/10; A61B 5/00; A61B 5/16; A61K 45/06

(52) U.S. Cl. CPC G16H 50/20; G16H 40/67; G16H 10/60; G16H 20/70; A61K 45/06; A61B 5/4839; A61B 5/165; A61B 5/4848; G16H 20/10

(57) ABSTRACT

A system for identifying amygdala hyperactivity in a human patient includes a network-connected mobile apparatus associated with the user. The network-connected mobile apparatus may be used to accept mood, energy and behavior input from the user and to accept third party patient observation data via the network. The network-connected device may be further used to accept provider-labeled patient observation data via the network and to provide behavioral suggestions to the user based on a set of profile rules. The profile rules may be generated from the behavior input from the user, the third-party patient observation data, and provider-labeled patient observation data as processed by a machine-learning system.

### CROSS-REFERENCE TO RELATED APPLICATIONS

This application claims the priority of prior Provisional U.S. Patent Application Ser. No. 63/069,013 filed on Aug. 22, 2020 and entitled “System and Method for Identifying Amygdala Hyperactivity in Human Patients,” which application is hereby incorporated by reference in its entirety.

### BACKGROUND

Since the passage of the U.S. Patient Protection and Affordable Care Act, also known as the Affordable Care Act or ACA, insurance providers have been required to cover addiction and mental health conditions in a similar manner to which they cover any other chronic disease. Many new treatment facilities are privately owned, stand-alone institutions without an integrated system to keep patients engaged past their inpatient stay. There has been a private equity surge in consolidating these centers; however, there are no overarching systems that unite and collaborate with these centers. During this time, the data on the relapse rates has been unable to be obtained due to poor and inconsistent reporting.

The transitions of care present within the current system represent points in treatment where patients are most likely to relapse, such as transitions between inpatient, partial hospitalization, intensive outpatient (IOP) services, and long-term aftercare. With the siloed approach and the increase in the number of treatment facilities competing for insurance dollars, there has been a loss of focus on continuity of care and patient outcomes. It is common practice for patients to be brought in from different cities for outpatient treatment and then sent back to their hometowns without supportive care or access to professional services. The current model encourages patients to attend Alcoholics Anonymous (AA) meetings; however, the patient is responsible for creating their own recovery support structure and community. Building a support structure community takes time, and without tools to help with this, patients are left without a support network.

### BRIEF DESCRIPTION OF THE DRAWINGS

The present disclosure is best understood from the following detailed description when read with the accompanying figures, wherein:

FIG. 1 is a block diagram of a diagnostic and treatment system according to one or more examples;

FIG. 2 is a diagram depicting of progression of localized seizure of the amygdala which begins with hyperactivity and progresses all the way to a diagnosis of temporal lobe epilepsy;

FIG. 3 is a diagram showing the transitions of care for a patient from inpatient to partial hospitalization to intensive outpatient treatment;

FIG. 4 is a flow diagram illustrating a methodology for assessing and treating a patient exhibiting anxiety;

FIG. 5 is a flow diagram illustrating a methodology for assessing and treating a patient exhibiting depression; and

FIG. 6 is a flow diagram illustrating an intervention protocol for treating a patient exhibiting anxiety or depression according to the methodologies of FIGS. 4 and 5.

It is emphasized that, in accordance with the standard practice in the industry, various features are not drawn to scale. In fact, the dimensions of the various features may be arbitrarily increased or reduced for clarity of discussion or illustration.

### DETAILED DESCRIPTION

At the IOP level of care there is limited or no access to physicians. Accordingly, patients may be told to establish relationships with their primary care provider (PCP). Without proper training, PCPs may place patients on medications that exacerbate their conditions. Patients may have access to physicians aware of the addiction; however, these services are often not utilized, due to lack of awareness of service availability and difficulty in accessing care. Due to this lack of continuity and a method by which patients who desire support are able to access it, there may be a gap in treatment at the different times in a patient's recovery. As is to be expected, some of these points in time may have a greater impact on whether or not comprehensive treatment will be successful.

Clinical experience has identified a subset of the population at high risk for relapse within the first days. Consistent clinical presentation of these patients paints a picture in which patients begin to experience an unremitting sense of terror, that they equate to anxiety, for a two- to seven-day period. Patients may not have any way to stop this discomfort and may relapse in a relatively short period of time. Clinical experience and preliminary data-gathering has been used to identify a progression in patient moods that has previously been unrecognized and uncharacterized.

Upon further examination and interviews it was revealed that the unremitting anxiety may be preceded by a characteristic escalation in patient energy levels, mood, and abilities to get work completed. This period of time was then followed by a period of difficulty completing simple tasks, confusion, paranoia and perseverating negative thoughts. This was then followed by what they described as unremitting terror and dread for a period of two to seven days. Many of these patients had been on several medications prior to these symptoms and none of them seem to help with the cycle. They are consistently stuck in a three-month cycle of relapse. Many of these patients would go out and use their drug of choice for two or three days and come back into the rooms of Alcoholics Anonymous and pick up a “desire chip,” symbolizing a desire to cease alcohol consumption for the next twenty-four hours.

To neurologists, localization of a lesion, such as of the amygdala, is of utmost importance when trying to diagnose a patient. Emotions of terror and fear localize to the amygdala and it has been determined that prior to the onset of the symptoms and escalation there was a trigger to evoke this core fear response. In particular, there may be a medical hyperactivity of the amygdala which in these patients which caused them to have the unremitting anxiety, terror and dread.

This anxiety that patients characterize was not alleviated by any known means including current meditation, talking with other people, going into a safe place, or attending a meeting. In one or more examples, a self-directed scale has been constructed to help determine a patient's energy level, overall wellness and motivation. These three accessories roughly correlated to the norepinephrine, epinephrine, serotonin and dopamine within their system. The patient's perceived energy levels related to the norepinephrine/epinephrine; the serotonin equated to their level of pleasant versus unpleasant and their level of activity correlated to their dopamine and motivation. Some patients had a variable upstroke in their energy levels and level of motivation as determined by the slope of the curve versus time and it is noted that the energy level increase precedes the activity level increase.

Certain medications have been found to exacerbate relapse cycles, and other medications were found to suppress relapse cycles. Difficulties that may arise with this patient population include patient inability to express feelings. Heretofore, there has not been shown a common language by which patients and physicians could share their experiences with the medications. Many times, patients may report feelings of depression initially; however, after careful questioning and clarification they describe symptoms that would be better characterized of hypomania. This inconsistent language and inability to articulate their internal state to the provider may lead to being placed on medications that either exacerbate the relapse cycle or do not allow for symptoms to be fully expressed. Without a shared language that is meaningful to both the providers and to use the patient's language, many patients may be caught in a cycle of being prescribed medications for which they do not know the effects. These patients may further be told to wait several months before coming back to clinic and determining whether or not there was efficacy.

Further, the experience of symptoms may not be accurately conveyed in between appointments unless patients are inventorying their moods on a daily basis. Patient recall bias may lead to a missed reporting of symptoms and incorrect reliance upon medications. Patients may only report what they are aware of and usually only report the times that are most distressing to them.

In one or more examples herein, a system referred to as “Mindful Medication” is implemented. In some examples, patients may be started on medications to help alleviate anxiety and instructed to take a small dose of the medication after assessing their own condition for a prescribed period (e.g., one minute or more) to determine where their symptoms of anxiety present. Patients may be instructed to record, such as via an app on a mobile device, the entire activity, and go through a stepwise process of checking with their bodies and becoming mindful of how they were feeling. Patients may be instructed to take half of a starting dose of their medications and check in with their bodies minutes later. In examples, this process of checking in with their feelings prior to taking medication allows for patients to become more attuned with what it is they were experiencing and give the provider more data as to what their preliminary symptoms were and how well the medication was doing within minutes after taking it. Due to the ego-centric nature of feelings, many patients may be unable to report how they were feeling a week ago, a day ago or even an hour ago without proper inventory. In examples, the system design allows patients and providers to become more attuned to the patient's feelings and have a consistent log by which the patient is checking in with their feelings. As used herein, the concept of the patient checking in with their “body” is similar to checking in with their “feelings.” However, checking in with the body may include a determination of physical manifestations of feelings in addition to emotional state.

In the treatment of Parkinson's disease, a wearable device such as kinetic watch with an accelerometer may be used to determine dyskinesia and states of high or low dopamine treatment levels. In examples herein, a similar methodology may be used to determine a patient's dopamine states to a subtle varying degree. Patients during high dopamine states are more likely to be motivated to do certain activities, have speed in motion, and normally are juggling multiple jobs at the same time and are moving at a faster rate. During low dopamine states patients may experience bradykinesia which can be measured on accelerometer and coupled with behavioral data. In examples, this may allow for a determination of the patient's level of attention and awareness during the states. Since it is suspected that accidents and injuries may be more likely to occur during high and low dopamine states, identifying these states may allow for increased levels of safety for patients who are fluctuating. This may then allow for correlation to be made between the wearable device with the information that the patient self-reports in an activities and mood log scale.

In examples, a plurality of subregions of the amygdala have been identified, each with different cellular morphology which leads to the different patterns of escalation. The compensatory behavior differs for these subregions. After determining that hyperactivity of the amygdala is causing an upstroke in anxiety symptoms and that the subsequent downstroke was a result of the burnout of the amygdala, a process of titrating them off the medications that was exacerbating the cycle may be initiated. This process may allow for tiny changes in medications to occur in a step-down fashion in patients being called in to clinic via telemedicine during optimum times of transition of medications. This slow gradual step down may allow for tiny changes to occur in the medications without causing an imbalance in patient feelings and a disruption with the potential of relapse.

As patients begin to change medications, it is possible that the frequency and amplitude of the slopes of the curve are attenuated by changes in medications. In some examples, a learning machine learning engine (or other artificial intelligence (AI) techniques, including but not limited to data mining, pathfinding, neural network programming, reinforcement learning, and multi-modal sentiment analysis), may be employed to become more and more attuned to the changes that are made among multiple patients, leading to better medications and timings being achieved for each individual patient. In addition to the timing of the medication changes, changes in medications may be made in a sequential fashion so as not to dysregulate the patient. As examples, taking off a dopamine blocker too early in the process may send the patient into a state of hypomania and leaving on a selective serotonin reuptake inhibitor (SSRI) for too long may exacerbate the situation as well. Medication changes may be made in a sequential fashion with the optimization of the process.

Patients may be most likely to relapse in zones and get into the most trouble during period of pleasant feelings and high energy. Many hypomanic symptoms present themselves during these periods of time and is important to monitor such symptoms. It may be unwise to change a patient's medications unless they are in close community and have a level of containment within their community and someone watching out for them. Therefore, in some examples, a system of checks and balances with an accountability partner is in order. The system may be provided in order to maintain patient safety when patients are not being monitored otherwise. It is also noted that oxytocin release is what most stabilizes the hyperactivity within the amygdala and is the forefront of treatment modality in some examples.

In examples, primary relapse criteria of unremitting negative thoughts with terror and dread which last for more than two or three days may be tracked and intervened upon. Patients experiencing this particular set of symptoms for longer than three days may be brought back immediately into clinic and treated with medications to stop and reset the progression. In some cases, this is a state in which patients relapse, with consequences up to and including suicide. With the system of monitoring and placement diagnostic mechanism according to one or more examples herein, it is possible to monitor and keep track of patients the possibilities of alleviating significant suffering.

Relapses tend to occur with the highest likelihood in periods of high energy and unpleasant feelings. Moving the patient and diminishing the movement from these states is the primary goal of treatment. Medications must be timed and changed in the appropriate manner. The fluctuation seen with addicts and alcoholics in early sobriety is a shift from pleasant to unpleasant with high energy levels. This is followed by a period of time in which they feel unpleasant with low energy. This triangular motion and pattern when treated will begin to diminish over time with the initial change being one in which the patient will move from unpleasant mood less and less frequently. As time goes on the fluctuation will diminish in a decrease in amplitude as we move the patient toward Euthymia.

Preferably, medications are changed with appropriate timing so that the maximum amount of compliance can be adhered to. Once a patient is able to feel what the medication effects are, there is a high likelihood of complying with the treatment modalities. Specifically, if a patient experiences a positive and timely reaction to a medication, they will be more inclined to continue that medication as prescribed.

Illustrative examples of the subject matter claimed below are disclosed. In the interest of clarity, not all features of an actual implementation are described for every example. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The amygdala is the localization of human emotional response. A patient's history of trauma has been correlated to the localized scarring in subsegments of the amygdala depending on the nature of the trauma. These scars affect the synchronicity and pathways by which neurons fire. Hypersensitive to external stimulus occurs in the amygdala and results in an increased perception of danger and threat. In severe cases, the asynchronous firing of these neurons results in a persistent hyperactivity that is not dampened by inhibitory signals from the prefrontal cortex. This hyperactivity is outwardly manifested as elevation in mood and changes in behavior. For those who suffer from substance use disorders (SUD), the inability to dampen the signal results in relapse to substance abuse.

Persistent amygdala asynchronous depolarization may be captured by mood, anxiety, energy levels, behavior tracking and accelerometers in wearable devices. The present disclosure, therefore, relates to a method and apparatus for treatment of mental health conditions involving a mobile app (and/or related wearable or IOT device) which captures the user's mood, anxiety, behavioral patterns (and the observations from the user's loved ones) and/or other sets of biomarkers with a high correlation to amygdala stimulation that can be used with machine learning to predict relapse events for those suffering from Substance Use Disorder.

Those who are long-term users of medications (e.g., benzodiazepines and suboxone) for a variety of behavioral disorders may experience increased mood and anxiety fluctuations when attempting to decrease their dosing. Various examples herein, therefore, relate to a method and apparatus for treatment of mental health conditions involving a mobile app (or related wearable or IOT device) which captures the user's mood and anxiety (and the observations from the user's loved ones) and/or other sets of biomarkers with a high correlation to amygdala hyperactivity that can be used with machine learning to aid in the tapering and titration of the addictive medications.

Bipolar disorder I, Bipolar II, and cyclothymia are disorders which may be difficult to treat. Patients may lack insight into their fluctuations between appointments and may be poor historians. Providers generally see patients on bi-monthly or monthly visit times resulting in incomplete data to make accurate medication titrations. Oftentimes medications are introduced in higher/lower dosages without a timely measure of response to medication effects. Some patients may perceive a blunting effect and have poor compliance with the regime. While other patients continue to have cycling moods due to under treatment or timing in which the medication was introduced. Allowing for small titrations of their medications and a slow introduction to these medications over the course of time would allow for better acceptance and compliance with medications.

In some examples herein, a system is provided to allow for machine learning and other artificial intelligence techniques as noted above to predict when a patient is experiencing an upswing. Determination of the upswing may allow for the prescriber to titrate within 24 hours of an upswing of the medications. This allows a patient to gradually be brought down to a euthymic state over the course of time with better compliance to medication as the patient becomes aware of the effects the medication is having on their mood.

It is also hypothesized that many of these mood fluctuations occur due to trauma induced scarring within the amygdala and that the best medications for these patients is a mood stabilizer/antiseizure medication which would allow for membrane stabilization and a gradual return to homeostasis. In some examples, a method and apparatus for treatment of mental health conditions is provided for involving a mobile application (or related wearable or IOT device) which captures the user's mood, energy level, behaviors, sleep patterns (and the observations from the user's loved ones) and/or other sets of biomarkers with a high correlation to daily mood. The application may be used with machine learning to aid in the titration of the medications used to treat bipolar disorder.

Anxiety disorders and OCD are also disorders that may be difficult to treat. Patients may lack insight into their behavior patterns and are poor historians. Providers generally see patients on bimonthly or monthly visit times resulting in incomplete data to make accurate medication titrations. In some examples herein, a system is provided to allow for machine learning to predict when a patient is experiencing increased periods of anxiety. It is also hypothesized that many of these mood fluctuations occur due to trauma within the amygdala and that the best medications for these patients is a mood stabilizer/antiseizure medication which would allow for dampening of the signals. In some examples herein, therefore, a method and apparatus for treatment of mental health conditions is provided involving a mobile application (or related wearable or IOT device) which captures the user's mood and anxiety (and the observations from the user's loved ones) and/or other sets of biomarkers with a high correlation to daily mood that can be used with machine learning to aid in the titration of the medications used to treat anxiety disorders and OCD.

Orthorexia Nervosa and Restrictive Anorexia Nervosa are also disorders that may be difficult to treat. Patients may lack insight into their behavior patterns and are poor historians. The excessive exercise exhibited by these patients may be an attempt to mitigate the hyperactivity of the amygdala and prevent the progression of the hyperactivity to feelings of confusion, paranoia and terror. In some examples herein, a system is provided to allow for machine learning to predict when a patient is experiencing increased periods of anxiety. It is also hypothesized that many of these mood fluctuations occur due to trauma within the amygdala and that the best medications for these patients is a mood stabilizer/antiseizure medication which would allow for dampening of the signals. In some examples herein, therefore, a method and apparatus for treatment of mental health conditions is provided involving a mobile application (or related wearable or IOT device) which captures the user's mood, anxiety, behavioral patterns (and the observations from the user's loved ones) and/or other sets of biomarkers with a high correlation to daily mood that can be used with machine learning to aid in the titration of the medications used to treat Othexia and Restrictive Anorexia.

Insomnia is a disorder that may be difficult to treat. Patients may lack insight into their behavior patterns and are poor historians. In some examples herein, a system is provided to allow for machine learning to predict when a patient is experiencing increased periods of anxiety, elevated mood and insomnia. It is also hypothesized that many of these mood, anxiety and insomnia symptoms occur due to trauma within the amygdala and that the best medications for these patients is a mood stabilizer/antiseizure medication which would allow for dampening of the signals. In some examples herein, therefore, a method and apparatus for treatment of mental health conditions is provided involving a mobile application (or related wearable or IOT device) which captures the user's mood, anxiety, behavioral patterns sleep patterns (and the observations from the user's loved ones) and/or other sets of biomarkers with a high correlation to daily mood that can be used with machine learning to aid in the titration of the medications used to treat insomnia.

In addition to psychiatric conditions, many of the chronic progressive diseases may be most effectively treated by calming the amygdala hyperactivity. It has been noted that patients manifest their stress in a variety of ways. Patients may identify conditions including, but not limited to, fibromyalgia, cluster migraine headaches, temporal lobe epilepsy and Irritable bowel syndrome. In some examples herein, a system is provided to allow for machine learning to predict when a patient is experiencing increased periods of anxiety. It is also hypothesized that many of these mood fluctuations occur due to trauma within the amygdala and that the best medications for these patients is a mood stabilizer/antiseizure medication which would allow for dampening of the signals. In some examples herein, therefore, a method and apparatus for treatment of mental health conditions is provided involving a mobile application (and/or related wearable or IOT device) which captures the user's mood, anxiety, behavioral patterns (and the observations from the user's loved ones) and/or other sets of biomarkers with a high correlation to daily mood that can be used with machine learning to aid in the titration of the medications used to treat/mitigate symptoms of Cluster Migraines, Irritable Bowel Syndrome, Fibromyalgia, and some Autoimmune disorders.

The highly vulnerable periods experienced by certain individuals, such as individuals in recovery from addiction, as well as patients diagnosed with other disorders, such as bipolar disorder, obsessive-compulsive disorder (OCD), insomnia, Orthexia and others, may be related to trauma induced mood fluctuation and cellular instability in the amygdala. These patients who exhibit extremes of this fluctuation describe a three-day period of time in which they have unremitting anxiety, which is not alleviated by any other known methods, including attending support group meetings (such as AA meetings), talking with their support group, taking medications, prayer and meditation.

These fluctuations appear to be most strongly noted in patients with a history of early childhood attachment trauma, sexual trauma, and history of violence witnessed or experienced. Once this experience begins, the instability to the amygdala hyperactive-medically referred to as an Affect or Sensory Seizure may be clinically localized. Young patients have clinically described the feelings of terror prior to their relapse. There is an older subset of the population which may also have similar experiences; however, due to neuroplastic changes in the brain, they have been observed to relapse in a much shorter duration from the onset of symptoms. Their family members are usually the first to identify this particular feeling.

The supportive system in place with twelve-step groups such as AA allow for daily reinforcement of prefrontal pathways which help to dampen amygdala hyperactivity. In addition, once the subject begins to trust the group, the hypothalamus is suspected to release oxytocin which also helps to stabilize the amygdala. Unfortunately, many patients that have severe trauma are unable to dampen the signals on their own early in sobriety and do not find long term sobriety.

It may be extrapolated that several other mood disorders, including depression, anxiety, schizophrenia and bipolar disorders, may originate in some patients with this amygdala hyperactivity and become heightened with repeated kindling. Other common diagnoses such as borderline personality disorder and obsessive-compulsive disorder (OCD) may be related to this same phenomenon to some degree.

It is also postulated that the current opioid crisis in the United States and elsewhere has been in response to patients using narcotics to alleviate hyperactivity of the amygdala. Systems in accordance with some examples herein may further be applied to the current estimate of millions American's prescribed benzodiazepine and have taken it for longer than one year In some examples herein, a system and method for behavior diagnosis from a combination of data from users, including from mobile applications, wearables, and Internet-of-Things (IoT) devices is provided.

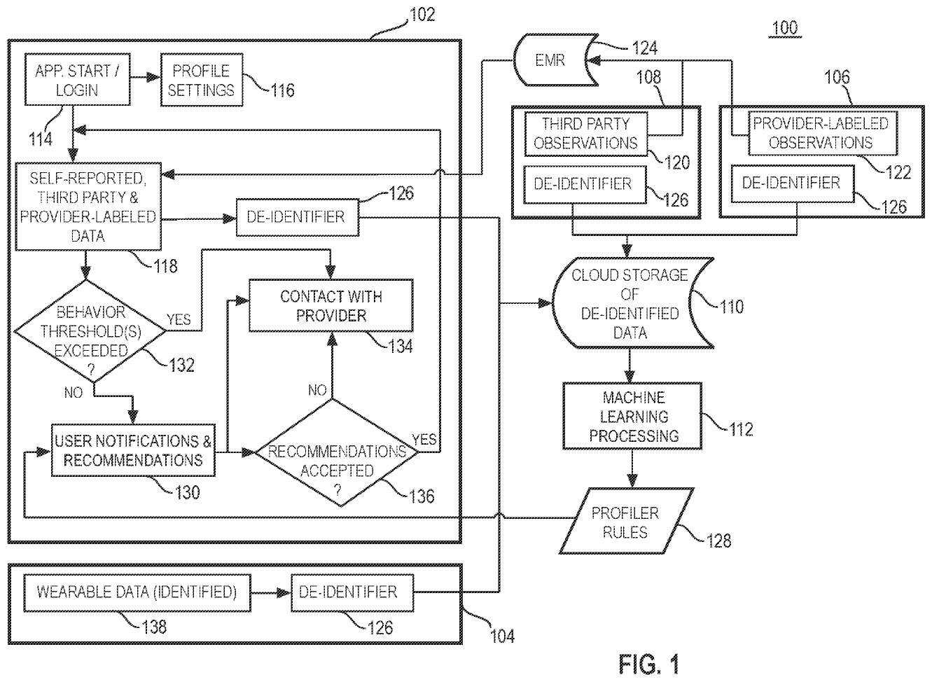

Referring now to FIG. 1, there is shown a functional block diagram of a treatment platform 100 according to one or more examples. As shown in FIG. 1, certain functionality of treatment platform 100 may be associated with and performed by a mobile device 102 associated with a patient. Mobile device 102 may be, for example, a smart phone capable of executing downloaded applications (“apps”).

Certain functionality of platform 100 may further be associated with and performed by one or more wearable or Internet-of-Things (IoT) devices 104 associated with a patient. Such wearable or IoT devices may include, for example, smart watches or other portable electronic devices, such as sleep tracking, weight tracking, or blood pressure monitoring devices. Device(s) 104 may be capable of wireless or direct communication with mobile device 102.

Further, certain functionality of platform 100 may be associated with and performed by medical providers 106, while other functionality of platform 100 may be associated with and performed by third parties 108, such as persons associated with the patient (e.g., friends, family, etc. . . . ).

Platform 100 may also include a cloud storage resource 110 for storing and providing access to patient-related data, as hereinafter described. In some examples, mobile device 102, wearable and IoT devices 104, medical providers 106 and third parties 108 may have access to cloud storage resource 110.

With continued reference to FIG. 1, platform 100 may also incorporate computational resources 112, which may be remotely located and which have access to cloud storage resource 110, as hereinafter described.

As noted, in some examples, mobile device 102 of a patient may execute one or more applications (“apps”) for implementing functionality of treatment platform 100. As shown in FIG. 1, in one example, this functionality may include a start/login function 114 for enabling a patient to provide and/or update patient profile settings 116 maintained in mobile device 102.

Functional block 118 in FIG. 1 represents a process of patient self-reporting on mood, anxiety status, and behavioral patterns. In some examples, a patient experience will, in part, gather his or her perception (his or her subjective experience) of his or her mood and anxiety on two dimensions with information about prior mood and anxiety levels when checking in. A distinction can be made between transient states of anxiety and those feelings of anxiety that are persistent. In some examples, an accompanying video presented on mobile device 102 may describe the differences.

“Anxiety and Mood” levels (and possibly a third dimension) define the level of energy and happiness that are self-described and along a spectrum of values (not a Boolean) in order to better risk stratify the user. In some examples, functionality represented by block 118 may include presentation of inquiries to the patient to elicit mood and anxiety status information.

In some examples, functionality of block 118 may include capturing of Hypomania Checklist (HCL-32) indicators. HCL-32 is a screening tool for identifying bipolarity in patients with depression, and includes a list of feelings and behaviors that often occur during hypomania. The following Table 1 is an example listing of factors which may be considered in capturing HCL-32 data:

TABLE 1
I need less sleep
I feel more energetic and more active
I am more self-confident
I enjoy my work more
I am more sociable (make more phone calls, go out more)
I want to travel and/or do travel more
I tend to drive faster or take more risks when driving
I spend more money/too much money
I take more risks in my daily life (in my work and/or other activities)
I am physically more active (sport etc.)
I plan more activities or projects.
I have more ideas, I am more creative
I am less shy or inhibited
I wear more colorful and more extravagant clothes/make-up
I want to meet or actually do meet more people
I am more interested in sex, and/or have increased sexual desire
I am more flirtatious and/or am more sexually active
I talk more
I think faster
I make more jokes or puns when 1 am talking
I am more easily distracted
I engage in lots of new things
My thoughts jump from topic to topic
I do things more quickly and/or more easily
I am more impatient and/or get irritable more easily
I can be exhausting or irritating for others
I get into more quarrels
My mood is higher, more optimistic
I drink more coffee
I smoke more cigarettes
I drink more alcohol
I take more drugs (sedatives, anti-anxiety pills, stimulants)

In some examples, the functionality represented by block 118 may further capture information as to activities that the user has engaged in. When inquiring, the app associated with block 119 may ask the patient such things as “What positive activities did you involve yourself in today?” (e.g., Prayer, meditation, going to support group meetings, calling his/her sponsor, reading literature, etc. . . . ). These include the anxiety reducing activities that the user was intending to complete.

With continued reference to FIG. 1, and as noted above, functional block 108 represents a process of acquiring information from third parties, such as friends and family of the patient.

In some examples, and if available, a third party (e.g., primary partner and/or the person who lives with the user) may provide additional data about the user, by being a user of a complementary app themselves. Observations of fluctuations of in a patient's mood and anxiety made by third parties, represented by block 120 in FIG. 1, may corroborate the self-reported data (block 118), and may potentially or precede self-reported observations it because the third parties may be aware of mood fluctuations before the patient, due to the patient's potential lack of awareness around his or her own behavior.

Similarly, functional block 106 represents a process of acquiring information from health providers who may be caring for the patient. Contact with a provider may happens in a variety of levels. Initial and immediate contact may be with a coach. The coach may arrange for treatment a therapist.

In some examples, a coach may use a script to educate the patient and to also capture information about the patient. The coach may provide additional resources to look and additional techniques to help calm down the patient's sense of anxiety and instruct the patient to contact a support group for help.

If patient anxiety persists despite having done intervention(s), the coach may schedule an appointment with a provider trained in trauma. The coach may collect additional data from the patient and set up a follow-up appointment.

Relevant provider labeled data, represented by block 122 in FIG. 1, may include:
Questionnaire to determine whether or not the patient has a history of trauma
Current medication regimen.
Length of sobriety.
Whether the cycles were happening previous to their use of app.
Provide more information to biopsychosocial.
Provide more information to medical history

As noted, provider information in block 122 may include information provided by a therapist. A licensed therapist who specialized in trauma may help the patient set priorities and goals of creating a healthy attachment and move them toward awareness and acceptance of how trauma is impacting their current disposition. Further, based upon recommendations from a therapist, a physician may titrate medications to ensure that hyperactivity was not potentiated by patient's current medications and cross taper them to stabilizing medications.

In some examples, care providers (e.g., doctors, coaches, therapists and the like) may provide and share identifiable, labeled patient data between one another through a separate electronic medical records (EMR) system, as represented by block 124 in FIG. 1.

On the other hand, in order to comply with patient privacy laws and regulations, such as the U.S. Health Insurance Portability and Accountability Act (HIPPA), various de-identifier processes 126 may be included in systems such as system 100 of FIG. 1. Each de-identifier block 126 functions to disassociate patient-identifying information such that, standing alone, any information processed by a de-identifier block 126 would not be able to be related to any particular patient, even if such information was intercepted, lawfully or otherwise, by a third party.

In the case of functional blocks 106 and 108 in the example of FIG. 1, de-identifier blocks 126 process provider-labeled observations (block 122) and third-party observations (block 120) may process patient information before such information is forwarded to a central database, such as a cloud storage database 110 of information. Cloud storage databased 110 may capture information from the various sources (patients, providers, third parties, and so on) within the treatment platform 100 of the example of FIG. 1.

As represented by block 112, a computational resource such as a machine-learning processing module, may be provided access to cloud storage 110 of de-identified information in order to process the de-identified patient information in order to develop and update a set of profiler rules 128 based upon the data and experiences not only of a given patient but of all patients participating in the treatment platform 100. Machine Learning processing 112 may be executed run on a scheduled basis to categorize users (patients) based on clustering of data then generate suggested therapeutic rules 128 and/or risk stratify the users.

In the generation of profiler rules 128, machine learning processing block 112 may employ clustering analysis based on instantaneous information and/or past information in order to generate predictive profiler rules having future diagnostic value to all users. In some examples, profiler rules 128 generalized through machine learning processing 112 may forwarded to a user recommendations module 130 executing on mobile device 102. User recommendations module 130 may provide user recommendations, such as by displaying messages on mobile device 102, in the form of suggestions and/or inspirational feedback intended to guide the user's behavior.

In some examples, user recommendations module 130 takes information from profiler rules block 128, applies the self-reported, third party, and provider data (past and present) from block 118 and generates a set of intervention(s). For example, recommendations module 130 may suggest an intervention which would change over time depending upon the self-reported data (past and present) and the output from the profiler rules. Suggestions would need to be “accepted” (block 136); otherwise, provider contact (block 134) would be advised.

In some examples, recommendations provided at block 130 may include recommendations of some positive behaviors to help reduce anxiety, include meditation, attending twelve-step meetings, calling a friend who is relaxing, prayer, taking anti-anxiety medication (preferably not habit-forming).

In some examples, user recommendations module 130 also receive input indicating whether any behavioral thresholds, as reflected in user self-reported data, third party data, or provider-labeled data, have been exceeded. In one example, an indication of unremitting anxiety (e.g., anxiety episodes occurring more than once per day), may be accounted for in treatment platform 100, as represented by block 132 in FIG. 1.

If, for example, in functional block 132 it is determined that a particular behavioral threshold has been exceeded, mobile device 102 may advise a user to contact his/her medical or therapeutic provider, as represented by block 134 in FIG. 1.

In block 136, treatment platform 100 may determine whether recommendations generated in block 130 have been accepted by a user. If a user does accept recommendations generated at block 130, this information may be fed back as self-reported data to block 118.

On the other hand, if in block 136 it is determined that a user has not accepted recommendations from block 130, this may also trigger advice to the user from block 134 to contact his/her medical/therapeutic provider.

As previously noted, in some examples, treatment platform 100 may further incorporate one or more user “wearable” devices 104. Wearable devices include such items as “smart watches” or other portable/wearable monitoring devices having sensors for measuring and assessing one or more user variables, such as heart rate, exercise levels, sleeping habits, and the like. Wearable device(s) 104 generate wearable data as represented by block 138 in FIG. 1. Such data may be user-identifiable, such that the data may be processed through de-identifier 126 prior to being provided to de-identified database 110.

In some examples, the present disclosure reflects that a total of nine nuclei are segmented for the amygdala, including lateral, basal, accessory-basal, anterior-amygdaloid-area (AAA), central, medial, cortical, cortical amygdaloid-transition (CAT), and paralaminar nucleus. Each of these subsegments leads to a different semiological presentation as the hyperactivity begins to expand across the amygdala, activating different subsegments. Each one of these subsegments has a different presenting behavior. This is contingent upon the first coping method which was used to compensate for the trauma which led to the scarring of the amygdala. Depending upon the degree of scarring and the repetitive nature of the scars, this may determine the velocity by which the expansion occurs and the speed in which behaviors present themselves. Over the course of a person's lifetime as the neuroplastic threads become reinforced, the progression by which the behavior changes will become subsequently more rapid as these regions are kindled and react faster. Being able to detect these patterns earlier in life will prevent the formation of reinforced grooves which lead to addictive behaviors as the progression of the coping mechanisms becomes more ingrained. The progressions through each of the subsegments is complex and unable to be identified through basic human observations. The observations have determined the predatory wounds, beginning in the basolateral amygdala, that are present in alcoholics and addicts. There is also an attachment wound which begins in the central amygdala which is common for those who present with anxious attachment styles.

As these progression are activated during times of emotional distress the summation and duration of the hyperactive will drive the behavior. This can be further predicted by computational analysis.

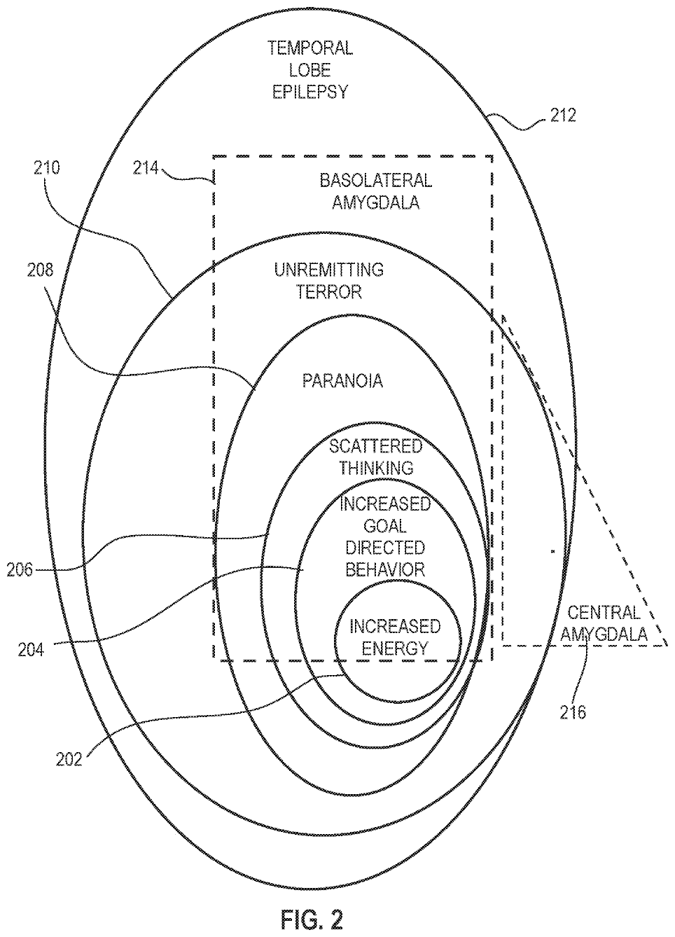

FIG. 2 is a diagram illustrating the progression of the hyperactivity from a potential nidus for hyperactivity to other parts of the basolateral amygdala. This progression has corresponding behavioral manifestations. Fear activates the progression illustrated in FIG. 2, in which element 214 represents the structure of the basolateral amygdala, which may be scarred due to trauma previously experienced by the patient. The progression starts with a region of increased energy in a first cluster of cells, represented by reference numeral 202 in FIG. 2, that produces increased levels of fight-or-flight neurotransmitters. The progression expands outward to a region of neighboring cells that are mediated by dopamine and lead to an increase in goal directed behavior, represented by element 204 in FIG. 2. A next phase of the progression moves to a region of cells associated with an uncoordinated hyperactivity of all of regions 202, 204, and 206 of FIG. 2 that behaviorally is expressed as scattered thinking. As the hyperactivity expands in the border zone between the basolateral amygdala 214 and the central amygdala (reference numeral 216 in FIG. 2), the patient may experience symptoms of paranoia (reference numeral 208). As the hyperactivity reaches and engulfs the central amygdala 216, patients may begin to express symptoms of unremitting terror (element 210). Finally, if the hyperactivity continues past the amygdala complex, the patient may present with temporal lobe epilepsy, as depicted by element 212 in FIG. 2.

Transitions of care have been demonstrated to be where the highest number of relapses occur. The patient is not as well monitored and supported in the outpatient setting as in an inpatient setting, and therefore would benefit from a system of care that allowed for access to a physician and monitoring of their mood fluctuations in the post-inpatient treatment phase of recovery. Therefore, in some examples, if a patient presents in a state of unrelenting terror, which may be sometimes interpreted as depression or hopelessness, or extreme anxiety, then a methodology designed to intervene in such states may be applied. Depending upon their presentation of either anxiety or depression, a different methodology may be employed to determine whether intervention will be beneficial. It may incumbent upon the provider to ascertain whether anxiety or depression is reported.

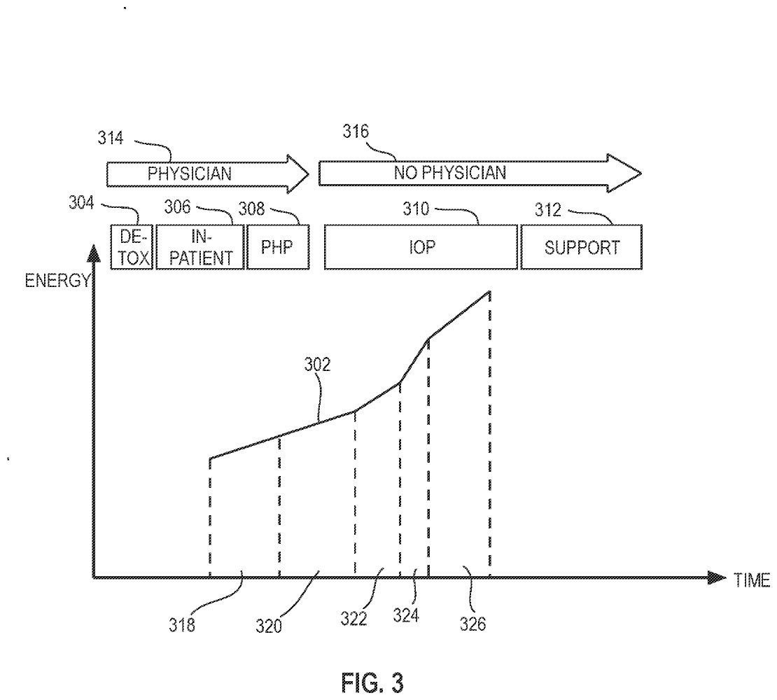

FIG. 3 is a diagram plotting energy (vertical axis) versus time (horizontal axis) and showing the transitions of care for a patient from detox inpatient treatment, partial hospitalization, intensive outpatient (IOP) treatment, and finally a support phase. The diagram of FIG. 3 helps to illustrate the high-risk areas for patients on discharge, particularly due to the typical lack of physician/provider care during critical phases of the recovery process.

A line 302 represents amygdala activity (energy) of a patent during a treatment cycle. FIG. 3 demonstrates that during the detox phase (reference numeral 304 in FIG. 3) and inpatient treatment (reference numeral 306), there is a low likelihood of escalation of patient energy (line 306). This may be attributed to the presence of physician care, the typical duration of which being represented by arrow 314 in FIG. 3, during these phases.

However, as the patient moves into the transitions of care between inpatient treatment 306 and partial hospitalization (reference numeral 308), and then from partial hospitalization 308 to IOP (reference numeral 310) and finally to a support phase (reference numeral 312), there may be little or no physician access or presence under typical treatment models, as represented by arrow 316 in FIG. 3.

As shown in FIG. 3, during treatment a patient may undergo increasing amygdala energy beginning with a period of increased energy (reference numeral 318 in FIG. 3), corresponding to region 202 of FIG. 2. As previously described with reference to FIG. 2, the ensuing phases of the progression may include a phase of increased goal directed behavior (reference numeral 320 in FIG. 3, corresponding to region 204 in FIG. 2), a phase of scattered thinking (reference numeral 322 in FIG. 3, corresponding to region 206 in FIG. 2), a phase of paranoia (reference numeral 324 in FIG. 3, corresponding to region 208 in FIG. 2) and a phase of unremitting terror (reference numeral 326 in FIG. 3, correspond to region 210 in FIG. 2).

As is evident from FIG. 3, the escalation of amygdala energy during a treatment process may occur during phases of treatment with limited or no physician presence (reference numeral 316). A treatment modality as described herein that provides for patient monitoring may facilitate recognition and intervention of the amygdala escalation exemplified by FIG. 3.

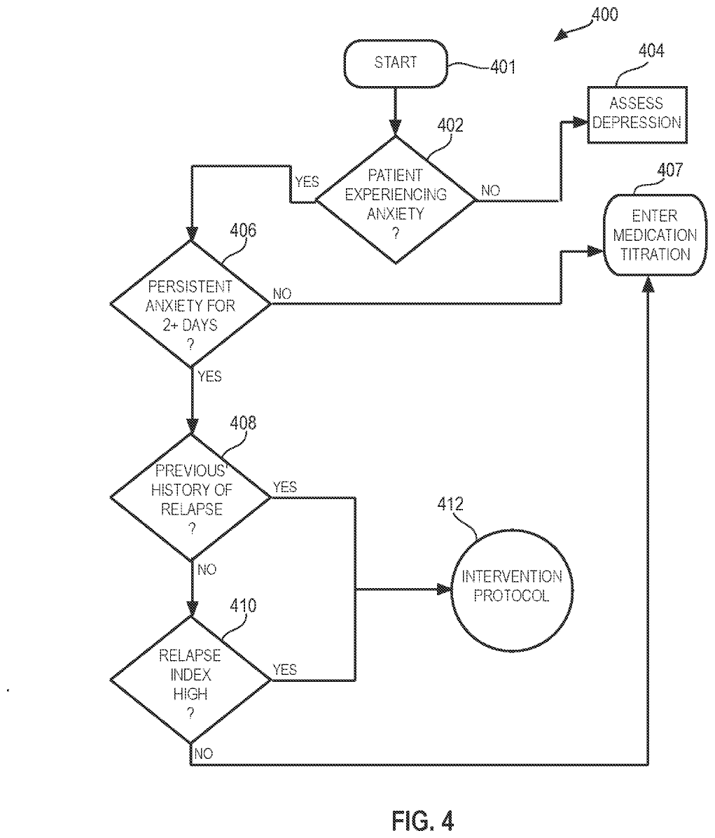

FIG. 4 is a flow diagram of a methodology 400 for treatment of a patient entering into a clinic (block 401) and presenting with a chief complaint of anxiety. In decision block 402, if the provider determines that the patient is experiencing anxiety, then in decision block 406 the provider may determine whether the anxiety has persisted for two or more days. Anxiety which is persistent and does not have any remittance to it may not be actual anxiety because it does not meet the requirements for shift in objective consciousness to allow for the anxiety to be dissipated. Thus, from block 406, if the anxiety has not persisted for over two days, a medication titration protocol may be entered, as represented by block 407 in FIG. 4.

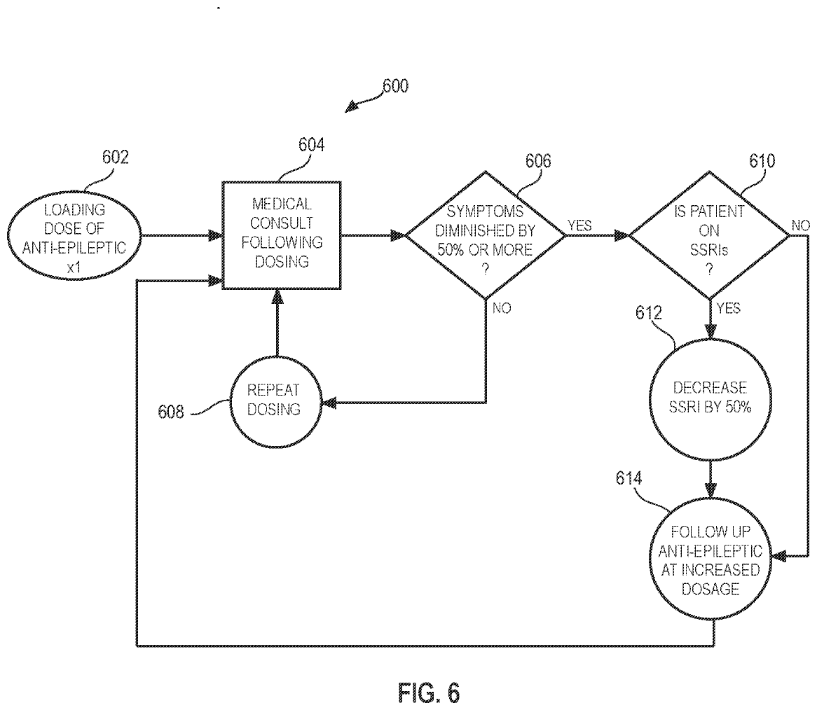

If the anxiety has persisted for two or more days without remittance, then in block 308, the provider may determine whether the patient has a history of relapse in the use of drugs or alcohol. If so, then the provider may proceed with the intervention protocol, as represented by block 412, and as described herein with reference to FIG. 6. If no history of relapse is shown, however, the provider may gauge the relapse anxiety index score for the patient. If it is high, as shown in FIG. 4, the provider may proceed with the intervention protocol, as represented by block 412.

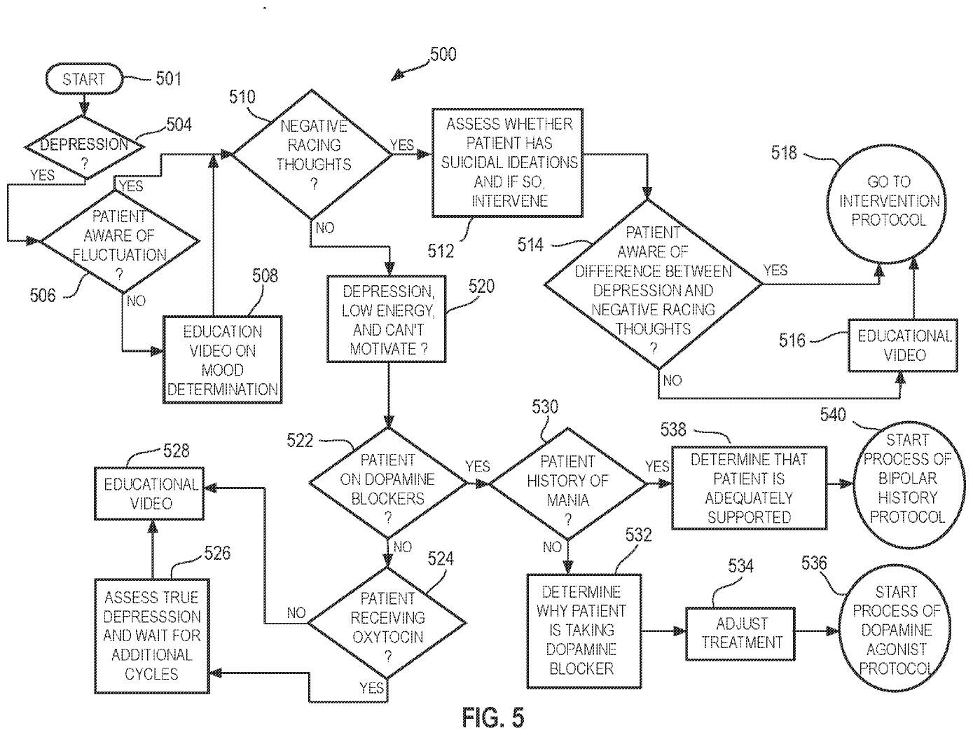

FIG. 5 is a flow diagram of a methodology 500 for treatment of progressions in FIGS. 2 and 3. If the patient does not present with anxiety as the chief complaint, the provider may rule out depression as the preliminary presenting event. Patients will present with either anxiety or depression, sometime complaining about them both at the same time. If the patient complains of depression, as determined in decision block 704, then, in decision block 506, an assessment may be made whether the patient is aware that they are cycling inside of their mood, and how aware they are of the ups and downs of the moods they are experiencing. If a patient is unaware of the fluctuations in block 506, education on mood-versus-feelings is indicated to bring them to such awareness, as represented by block 508.

On the other hand, in block 504, if the patient is aware of the fluctuations, a provider may investigate whether depressive symptoms are truly depressive. This begins in decision block 510, wherein a determination is made as to whether the patient is experiencing negative racing thoughts. If the depression exhibits as negative racing thoughts, then, in block 512, an assessment may be made whether the patient has suicidal ideations. If so, appropriate immediate intervention is indicated.

Assuming no suicidal ideations are exhibited in block 512, then in decision block 514, a determination is made whether the patient is aware that depression and negative racing thoughts are not the same. If the answer in block 514 is yes, then at block 518 the provider may proceed to the intervention protocol (described herein with reference to FIG. 6). If the answer in decision block 514 is that the patient is unaware of the difference between depression and negative racing thoughts, then the patient may be provided an educational video or other educational modalities to explain this distinction and bring the patient the awareness that negative racing thoughts is a hypomanic state, as represented by block 516 in FIG. 5, after which the provider may initiate the intervention protocol, as represented by block 518.

Returning to decision block 510, if the patient's depressive symptoms are not negative racing thoughts, an assessment may be made in block 520 whether the patient is experiencing a true depressive state in which the patient has low energy and cannot motivate. If so, the next question, in decision block 522, is whether the patient is currently taking dopamine blockers, such as, without limitation aripiprazole or ziprasidone. If so, as represented by block 530, a determination may be made whether the patient has a history of mania/manic behaviors. If so, as represented by block 538, a provider must ensure that the patient has appropriate and adequate counseling and support, as represented by block 538, and should initiate a process/protocol for addressing bipolar disorders, as represented by block 540.

On the other hand, in block 530 if it is determined that the patient does not have a history of mania/manic behaviors, then in block 532, a determination may be made why the patient is taking dopamine blockers, and in block 534, appropriate adjustments to the dopamine blocking treatment may be made before initiating a dopamine agonist protocol, as represented by block 536.

In block 522, if it is determined that the patient is not taking a dopamine blocker, then, in block 524, a determination is made whether the patient is receiving oxytocin, such as through attendance at twelve-step meetings and with fellowship and interaction in recovery communities. If so, in block 526, an assessment of true depression may be made, and the provider may wait for further manifestations in order to address these symptoms. In either case, the patient may be provided, in block 528, with educational videos or other educational modalities to increase the patient's understanding of his or her state.

If it has been determined that a patient is to be intervened upon, according to the methodologies described herein with reference to FIGS. 4 (anxiety) and 5 (depression), a provider may enter into the intervention protocol 600 of FIG. 6, beginning at block 602 with the administration of a loading dose of an anti-epileptic medication such as, for example but without limitation, Depakote® (divalproex), Lamictal® (lamotrigine), or Oxtellar XR®/Trileptal® (oxicarbamazepine). This may be followed by a telemedicine (or in-person) medical consult, as represented by block 604, some period of time following administration of the loading dose. This period of time may vary from a few hours to a day or two.

From this consult, the provider may ascertain from the patient, in block 606, whether the symptoms have diminished by 50% or more. If symptoms have not sufficiently diminished, in block 608, a repeat dosing of the anti-epileptic may be administered, and the process repeated.

On the other hand, if the loading dose does achieve a 50% or greater diminution of symptoms in block 606, then in block 610, a determination is made whether the patient is taking selective serotonin reuptake inhibitor (SSRI) If so, then, in block 612, this medication may be decreased by 50% and, in block 614, a follow-up of the anti-epileptic medication at an increased dosage may be administered. If in block 610 it is determined that the patient is not taking SSRI medication, then the increased dosage of anti-epileptic medication is administered in block 614. Following administration of the increased dosage in block 614, a follow-up medical consult may be conducted and the process repeated.

As used herein, the article “a” is intended to have its ordinary meaning in the patent arts, namely “one or more.” Herein, the term “about” when applied to a value generally means within the tolerance range of the equipment used to produce the value, or in some examples, means plus or minus or plus or minus 5%, or plus or minus 1%, unless otherwise expressly specified. Further, herein the term “substantially” as used herein means a majority, or almost all, or all, or an amount with a range of about 51% to about, for example. Moreover, examples herein are intended to be illustrative only and are presented for discussion purposes and not by way of limitation.

In this description, for purposes of explanation, numerous specific details are set forth in order to provide a thorough understanding of the examples disclosed herein. It will be apparent, however, to one skilled in the art that the disclosed example implementations may be practiced without these specific details. In other instances, structure and devices are shown in block diagram form in order to avoid obscuring the disclosed examples. Moreover, the language used in this disclosure has been principally selected for readability and instructional purposes and may not have been selected to delineate or circumscribe the inventive subject matter, resorting to the claims being necessary to determine such inventive subject matter. Reference in the specification to “one example” or to “an example” means that a particular feature, structure, or characteristic described in connection with the examples is included in at least one implementation.

The terms “computing system” and “computing resource” are intended broadly to refer to at least one electronic computing device that includes, but is not limited to including, a single computer, virtual machine, virtual container, host, server, laptop, and/or mobile device, or to a plurality of electronic computing devices working together to perform the function(s) described as being performed on or by the computing system or computing resource. The terms also may be used to refer to a number of such electronic computing devices in electronic communication with one another, such as via a computer network.

The term “computer processor” is intended broadly to refer to one or more electronic components typically found in computing systems, such as microprocessors, microcontrollers, application-specific integrated circuits (ASICS), specifically-configured integrated circuits, and the like, which may include and/or cooperate with one or more memory resources, to perform functions through execution of sequences of programming instructions.

The terms “memory” and “memory resources” are intended broadly to refer to devices providing for storage and retrieval of data and programming instructions, including, without limitation: one or more integrated circuit (IC) memory devices, particularly semiconductor memory devices; modules consisting of one or more discrete memory devices; and mass storage devices such as magnetic, optical, and solid-state “hard drives.” Semiconductor memory devices fall into a variety of classes, including, without limitation: read-only-memory (ROM); random access memory (RAM), which includes many sub-classes such as static RAM (SRAM), dynamic RAM (DRAM), non-volatile RAM (NVRAM), and others; electrically-alterable memory; flash memory; electrically-erasable programmable read-only memory (EEPROM), and others.

The term “non-transitory storage medium” is intended broadly to include any and all of the above-described forms of memory resources, and one or more such resources, comprising physical, tangible storage media that store the contents described as being stored thereon.

The term “cloud,” as in “cloud computing” or “cloud resource,” refers to a paradigm that enables ubiquitous access to shared pools of configurable computing resources and higher-level services that can be rapidly provisioned with minimal management effort; often, cloud resources are accessed via the Internet. An advantage of cloud computing and cloud resources is that a group of networked computing resources providing services need not be individually addressed or managed by users; instead, an entire provider-managed combination or suite of hardware and software can be thought of as an amorphous “cloud.”

The terms “application,” “function,” and “module” refer to one or more computing, programs, processes, workloads, threads and/or sets of computing instructions executed by a computing system, and to the computing hardware upon which such instructions may be performed. Example implementations of applications, functions, and modules include software modules, software objects, software instances and/or other types of executable code. The use of the term “application instance” when used in the context of cloud computing is intended to refer to an instance within the cloud infrastructure for executing applications (e.g., for a resource user in that user's isolated instance).

Any application, function, module described herein may be implemented in various hardware arrangements and configurations to embody the operational behavior of the application, function, or module described. As a non-limiting example, an application, function, or module may be implemented in hardware including a microprocessor, microcontroller, or the like, incorporating or cooperating with program storage hardware embodying instructions to control the hardware to operate as described. As another non-limiting example, an application, function, or module may be implemented in hardware including application-specific integrated circuitry (ASIC) tangibly embodying the function of such application, function, or module as described.

The term “machine learning,” which may be considered a sub-category of artificial intelligence, refers to algorithms and statistical models that computers and computing systems use to perform specific tasks without using explicit instructions, instead relying on models, inference and other techniques. Machine learning is considered a subset of the broader field of artificial intelligence. “Machine-learned algorithms” are algorithms which generally involve accepting and processing input data according to a desired function and/or to generate desired output data. The desired function of a machine-learned algorithm, typically implemented by a computer processor, is established by using one or more sample datasets, known as “training data,” to effectively “program” the processor to perform the desired function. Thus, machine-learned algorithms enable a processor to perform tasks without having explicit programming to perform such tasks.

For example, if a desired task is to recognize the presence of a particular data pattern within a given data object, the training data for a machine learning algorithm may include data objects known to be with and without the pattern to be recognized. Once trained, a system including a processor implementing the machine-learned algorithm takes an unknown dataset as input, and generates an output or performs some desired function according to its training. In the foregoing pattern recognition example, application of the machine-learned algorithm on a data object (the input to the algorithm) may cause the data object to be classified according to whether or not the pattern was recognized in the data object. In this example, classification of the input data object according to the training of the algorithm constitutes the desired output of the machine-learned algorithm.

In various examples herein, a user interface/user experience treatment platform is provided that captures self-reported data on a mobile device to identify an unremitting amygdala hyperactivity.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

What is claimed is:

1. A system for identifying amygdala hyperactivity in a human patient, comprising:
a network-connected mobile apparatus associated with the user, the mobile apparatus including a processor for executing an application for:
accepting mood, energy and behavior input from the user;
accepting third party patient observation data via the network;
accepting provider-labeled patient observation data via the network;
providing behavioral suggestions to the user based on a set of profile rules;
wherein the profile rules are generated from the behavior input from the user, the third-party patient observation data, and provider-labeled patient observation data as processed by a machine-learning system.

2. The mobile apparatus of claim 1, wherein the profile rules include rules for identifying and predicting amygdala hyperactivity in the user.

3. A network-connected mobile apparatus associated with a user, the mobile apparatus including a processor for executing an application for:
accepting mood, energy and behavior input from the user;
accepting third party patient observation data via the network;
wearable devices;
accepting provider-labeled patient observation data via the network;
providing behavioral suggestions to the user based on a set of profile rules;
providing medication timing and effects inputs from patients;
wherein the profile rules are generated from the behavior input from the user, the third-party patient observation data, wearable devices and provider-labeled patient observation data as processed by a machine-learning system.

4. The mobile apparatus of claim 3, wherein the profile rules include rules for treating amygdala hyperactivity in the used.

5. A method for treatment of a human patient recovering from substance addiction, comprising:
when the patient is exhibiting symptoms of persistent anxiety for over two days, and has a previous history of relapse, initiating an intervention protocol comprising:
administering a loading dose of an anti-epileptic medication;
providing a medical consult with the patient within a predetermined period of time following the administration of the loading dose of anti-epileptic medication to determine whether the symptoms of persistent anxiety have decreased by at least fifty percent;
wherein if the symptoms of persistent anxiety have not decreased by at least fifty percent, administering a further dose of the anti-epileptic medication;
and wherein if the symptoms of persistent anxiety have decreased by at least fifty percent, decreasing any selective serotonin reuptake inhibitor medication being taken by the patient by fifty percent, and administering a further dose of the anti-epileptic medication.

6. A method for treatment of a human patient recovering from substance addiction, comprising:
when the patient is exhibiting symptoms of depression persistent anxiety for over two days, initiating an intervention protocol comprising:
administering a loading dose of an anti-epileptic medication;
providing a medical consult with the patient within a predetermined period of time following the administration of the loading dose of anti-epileptic medication to determine whether the symptoms of persistent anxiety have decreased by at least fifty percent;
wherein if the symptoms of persistent anxiety have not decreased by at least fifty percent, administering a further dose of the anti-epileptic medication;
and wherein if the symptoms of persistent anxiety have decreased by at least fifty percent, decreasing any selective serotonin reuptake inhibitor medication being taken by the patient by fifty percent, and administering a further dose of the anti-epileptic medication.
